(19) United States
(12) Patent Application Publication
(10) Pub. No.: US 2005/0250117 A1
(43) Pub. Date: Nov. 10, 2005

(54) Isolation of single polymeric molecules

(76) Inventors: Xing Su, Cupertino, CA (US); Narayan Sundararajan, San Francisco, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/959,237

(22) Filed: Oct. 5, 2004

Related U.S. Application Data
(63) Continuation-in-part of application No. 10/748,802, filed on Dec. 30, 2003.
(60) Provisional application No. 60/509,707, filed on Oct. 7, 2003.

(51) Int. Cl. C12Q 1/68; C07H 21/04

(52) U.S. Cl. 435/6; 536/25.4

(57) ABSTRACT

Methods and devices for immobilizing and isolating single polymeric molecules are disclosed. In some aspects, controllable dispensing of target polymeric molecules is provided. In additional aspects of the invention, methods for creating and manipulating microbeads having a single target nucleic acid molecule attached are provided. Aspects of the disclosed devices and methods are exemplified using microfluidics.

### CROSS-REFERENCE TO RELATED APPLICATIONS

The present application is a continuation-in-part of U.S. application Ser. No. 10/748,02, filed Dec. 30, 2003, which in turn claims the benefit under 35 U.S.C. §119(e) of U.S. Provisional Patent Application No. 60/509,707, filed Oct. 7, 2003, the disclosures of which are incorporated herein by reference.

### TECHNICAL FIELD

The disclosure is generally related to devices and methods for immobilizing, isolating, manipulating, and controllably dispensing individual polymeric molecules.

### BACKGROUND

Single molecule analysis is of interest for a variety of reasons, including its potential for providing high-resolution information for individual genotypes. Such sequence information may be used, for example, to identify genetic variations that cause or contribute to disease states and/or to increase pharmaceutical efficacy. Additionally, the sensitive and accurate detection, isolation, and identification of single molecules from biological and other samples has widespread application in medical diagnostics, pathology, toxicology, environmental sampling, chemical analysis, forensics and numerous other fields. Generating the desired information, however, essentially requires that a single targeted DNA molecule be physically isolated from a complex mixture and manipulated in a manner that permits subsequent analysis. Isolating and manipulating single molecules is technically challenging.

Polymeric molecules, such as DNA, having various functional groups attached to their ends have been attached to solid supports by covalent bonds formed between the attached functional groups and complementary groups present on the solid support surface. For example, single DNA molecules covalently attached to beads have been manipulated with optical tweezers (T. Perkins et al., Science, 264: 822-826 (1994)). Polymeric molecules have been hybridized to complementary molecules covalently attached to a substrate, and subsequently released from the substrate. For example, infrared laser irradiation has been used to thermally denature and release DNA molecules immobilized to specific areas of a conventional DNA chip (K. Okano et al., Sensors and Actuators, 64:88-94 (2000)).

### BRIEF DESCRIPTION OF THE DRAWINGS

FIG. 1(A) illustrates a portion of a substrate to which blocking agents and a binding agent have been attached. An oligo dT (oligonucleotide) attached to a binding partner is attached to the binding agent through a binding agent-binding partner linkage. FIG. 1(B) illustrates the modification of a target double-stranded DNA molecule (dsDNA) with a poly dA tail and a synthetic capping oligonucleotide (a hairpin-like oligonucleotide) containing a functional group.

FIG. 2 illustrates devices and a procedure for isolating a polymeric molecule according to an aspect of the present invention. In FIG. 2(A), a target oligonucleotide is hybridized to a substrate at a binding position. In FIG. 2(B) a microsphere is attached to the isolated target oligonucleotide. In FIG. 2(C), the microsphere having a single attached DNA molecule is released from the substrate.

FIG. 3 illustrates the positioning of polymeric molecules (such as DNA) on a substrate such that the shortest distance between the molecules is at least two times the length of the individual polymeric molecules.

FIG. 4 illustrates a procedure for labeling DNA molecules.

FIG. 5 illustrates the attachment of biotin-labeled single-stranded (ssDNA) to a microsphere bead functionalized on its surface with streptavidin.

FIGS. 6A-D illustrate devices and a procedure for attaching a single polymeric molecule to a microsphere bead.

FIG. 7 illustrates the predicted dependence of melting temperature on the length of the hybridized DNA oligonucleotide.

FIG. 8 illustrates an apparatus for selective heating of a substrate according to an aspect of the present invention. In FIG. 8(A), resistive heating of the center electrode causes the oligonucleotides hybridized above the electrodes to detach from the surface. In FIG. 8(B), resistive heating of the outer electrodes causes the oligonucleotides hybridized above the outer electrodes to detach from the surface.

FIG. 9(A) illustrates a glass substrate containing patterned resistive heating elements. FIG. 9(B) illustrates the positioning of a microchannel relative to the resistive heaters. FIG. 9(C) illustrates a view of a microchannel positioned above the patterned resistive heating elements.

FIG. 10 illustrates a microfluidic device in accordance with one embodiment of the invention and includes an expanded, projected view of an exemplary microfluidic device and substrate.

FIG. 11 is a cross-sectional view along line 2-2 of the microfluidic device depicted in FIG. 10.

FIG. 12 illustrates a microfluidic device in accordance with an additional embodiment of the invention.

### DETAILED DESCRIPTION OF THE INVENTION

In general, molecules useful in the present invention include polymers of deoxyribonucleotides or ribonucleotides and analogs thereof that are linked together by a phosphodiester bond. A polynucleotide can be RNA or DNA, and can be a gene or a portion thereof, a cDNA, a synthetic polydeoxyribonucleic acid sequence, or the like. In various embodiments, a polynucleotide, including an oligonucleotide (for example, a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides.

The covalent bond linking the nucleotides of a polynucleotide generally is a phosphodiester bond. However, the covalent bond also can be any of a number of other types of bonds, including a thiodiester bond, a phosphorothioate bond, a peptide-like amide bond or any other bond known to those in the art as useful for linking nucleotides to produce synthetic polynucleotides. The incorporation of non-naturally occurring nucleotide analogs or bonds linking the nucleotides or analogs can be particularly useful where the polynucleotide is to be exposed to an environment that can contain nucleolytic activity, since the modified polynucleotides can be less susceptible to degradation.

Virtually any naturally occurring nucleic acid may be prepared and manipulated by the disclosed methods including, without limit, chromosomal, mitochondrial or chloroplast DNA or ribosomal, transfer, heterogeneous nuclear or messenger RNA. Nucleic acids may be obtained from either prokaryotic or eukaryotic sources by standard methods known in the art. RNA can be converted into more stable cDNA through the use of a reverse transcriptase enzyme. Methods for preparing and isolating various forms of nucleic acids are known. (See for example, Berger and Kimmel, eds., Guide to Molecular Cloning Techniques, Academic Press, New York, N.Y. (1987); Sambrook, Fritsch and Maniatis, eds., Molecular Cloning: A Laboratory Manual, 2nd Ed., Cold Spring Harbor Press, Cold Spring Harbor, N.Y. (1989).) However, embodiments of the present invention are not limited to a particular method for the preparation of nucleic acids.

In one embodiment of the present invention, methods and devices are provided for immobilizing and isolating single nucleic acid polymer molecules on a substrate. This isolation and immobilization allows a nucleic acid molecule to be controllably released into solution. In this manner, it is possible to isolate and selectively dispense single molecules for subsequent single molecule analysis.

According to an aspect of the present invention, polymeric molecules, such as nucleic acids, are immobilized on substrates. The number of polymeric molecules immobilized on a substrate can be controlled by providing regions on the substrate that have areas to which the polymeric molecule may bind and areas to which the polymeric molecule does not bind. Such isolation and immobilization allows the polymeric molecules to be further manipulated, such as through the attachment of a microsphere bead. Target polynucleotide molecules can then be released from the substrate surface. Target molecule release can be controlled as disclosed herein.

In general, suitable substrates for embodiments of the present invention include, for example, gold substrates, aluminum substrates, glass substrates, silicon substrates, and polymeric substrates such as poly(methyl methacrylate) and poly(dimethyl siloxane). Furthermore, the substrate can be any metal layer or any organic polymer layer that can be modified to provide binding positions. Prior to immobilizing polymeric molecules thereto, the substrate typically is modified to include binding positions. For example, a suitable glass substrate can be treated with sodium hydroxide to expose reactive, hydroxyl groups. The hydroxyl groups of the substrate can be further reacted with an aldehyde-containing silane reagent to form an aldehyde-activated substrate. Aldehyde-activated substrates are commercially available (NoAb BioDiscoveries Inc., Ontario, Canada). Suitable substrates can alternatively be reacted with silane reagents containing carboxyl groups, amino groups, and/or epoxy groups to form carboxyl-activated substrates, amino-activated substrates, and/or epoxy activated substrates.

The activated substrate can then be treated with a mixture comprising a binding agent and a non-binding blocking agent. A binding agent is a molecule or atom that can form a strong interaction with a polymeric modification. For example, gold forms a covalent binding interaction with thiol-modified polymer molecules; antibodies are available which selectively bind such molecular labels as fluorescein and digoxigenin or cholesterol, and avidin and streptavidin have a non-covalent binding interaction with biotin with an energy equivalent to some covalent bonds. The term antibody as used herein includes polyclonal and monoclonal antibodies as well as fragments thereof, recombinant antibodies, chemically modified antibodies and humanized antibodies, all of which can be single-chain or multiple-chain. Further examples of binding partners include protein-aptamer, lectin-sugar (and lectin-carbohydrate), and enzyme-inhibitor cofactor, or substrate interactions.

A functional non-binding agent or a blocking agent is a molecule or atom which does not form a strong interaction with a polymeric modification. For example, platinum (Pt) and copper (Cu) do not have a binding interaction with thiol groups; a carboxy-modified substrate will not bind to thiol modified polymers; bovine serum albumin (BSA) and bovine IgG (BIgG) do not have a binding interaction with biotin; and streptavidin or avidin do not bind to digoxigenin.

For example, according to one exemplified embodiment of the invention, an aldehyde-activated substrate is reacted with a mixture comprising a receptor and bovine serum albumin (BSA). The aldehyde functional groups of the substrate react with amines present on the proteinaceous receptors and blocking agents to form covalent bonds, thereby attaching the receptors and the blocking agents to the substrate. The attached receptors provide individual localized precursor binding positions on the substrate.

The precursor binding positions created by the binding agents on the substrate can be further modified by attaching oligomeric or polymeric molecules to provide binding positions. For example, an oligonucleotide having a 5′ end labeled with a binding partner can be reacted with the binding positions on the substrate. Preferably, the binding agent has binding affinity for the oligonucleotide label. Although illustrated in the examples as an oligo-dT, other sequences may be chosen. Typically, the sequence chosen will be one that is complementary to and capable of hybridization to a portion of the target polymeric molecule. For attachment to a binding agent, a particular polymer molecule can be modified, for example, with a thiol group or a biotin group. Modification allows the polymer molecule to be attached at one end through a particular type of attachment, for example, a thiol group/gold interaction or biotin/avidin (or streptavidin) interaction.

Modifications to a target polymer molecule may include any chemical functional group as well as standard molecular labeling techniques. The particular type of modification is chosen to maximize its binding potential with the specific binding agent and minimize its potential for binding to the functional non-binding agent or the surface of the support material used in the disclosed methods and devices. Polymers can be modified with labels or tags that are commonly used in the art. For nucleic acids such labels include, but are not limited to, biotin, fluorescein, digoxigenin, and the like. Further examples of binding partners include protein-aptamer, lectin-sugar (and lectin-carbohydrate), and enzyme-inhibitor, cofactor, or substrate interactions. Such modifications are well known in the art and commercial nucleic acid synthesis vendors provide such modification services (for example Qiagen-Operon, Valencia, Calif.).

Alternatively, a suitable substrate can be modified to include binding positions separated by at least about two times the length of the polymeric molecules by treating the substrate surface with a mixture comprising a functionalized oligomer and a blocking agent. For example, a gold substrate can be treated with mixture including a thiol-modified nucleic acid oligomer (functionalized oligomer) and hexadecanethiol (blocking agent). The mixture ratio of functionalized oligomer to blocking agent can be about one to about ten. Alternatively, the ratio can also be about one functionalized oligomer to about 10,000 blocking agents, one functionalized oligomer to about 100,000 blocking agents, one functionalized oligomer to about 1,000,000 blocking agents, and about one functionalized oligomer to about 10,000,000 blocking agents. Again, other suitable ratios can be determined by one having ordinary skill in the art and as disclosed herein. Substrate binding positions separated by at least about two times the length of the polymeric molecules can be also provided by treating the activated substrate first with a solution containing the functionalized oligomer and subsequently with a solution comprising the blocking agent.

The attached oligomers (for example, oligonucleotides) of the binding positions are contacted with target polymeric molecules. Target labeled polymeric molecules that are complementary to the attached oligomers hybridize to the attached oligomers and are thereby immobilized at the binding positions on the substrate. According to one embodiment of the invention, an oligomer is synthesized to be complementary to a specific known region in a target polymeric molecule. Alternatively, an attached oligomer can be synthesized to be complementary to a specific region in a target molecule which has been added or ligated to the target molecule.

Target molecules can be prepared, for example, by digesting a DNA sample with two different restriction enzymes to create DNA fragments with two different ends. In one aspect, a hairpin-like oligonucleotide containing a biotin moiety in the middle and a restriction enzyme site or at least an appropriate overhanging end at its end is ligated to one desired end of the digested DNA, and an oligonucleotide, which is designed to be complementary to the binding site oligomer on the substrate, can be added to the other end. The digested DNA sample can also be treated with a polymerase to provide a tail (for which the sequence is known by virtue of controlled polymerization, for example, poly-dT-tail) for potential hybridization to the substrate binding site oligomer.

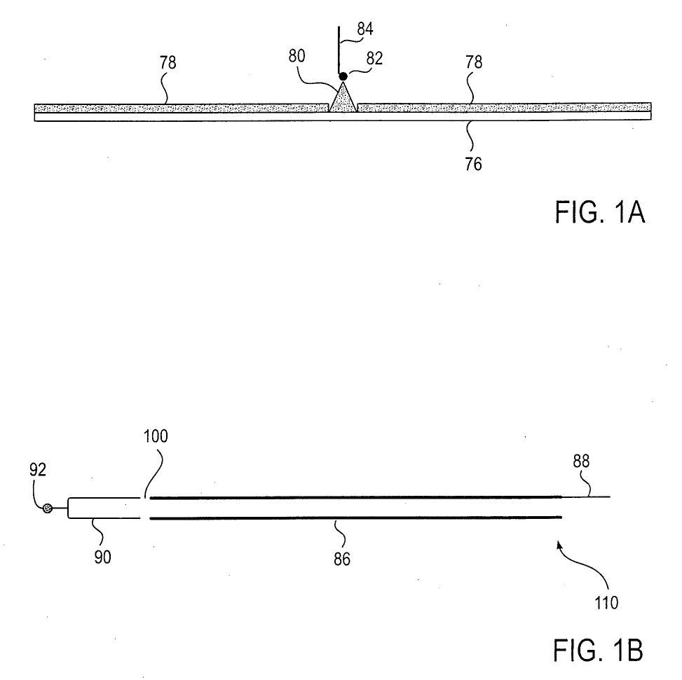

Referring now to FIG. 1, an illustration is provided of a device that allows polymeric molecules to be immobilized according to an aspect of the invention. In FIG. 1A, a substrate 76 is coated with a non-binding agent 78 and a binding agent 80 to create a surface to which polymeric molecules can be bound. Also shown in FIG. 1A, an oligo-dT 84, having an attached binding partner 82, has been attached to the site created by the binding agent 80 through a binding agent-binding partner linkage thus creating an attachment site for a polymeric molecule having a linked oligo-dA. In FIG. 1B, a double-stranded DNA molecule (dsDNA) 86 according to an aspect of the present invention having modifications on both ends is shown. In FIG. 1B, the modified dsDNA 110 contains a poly dA tail 88 and a synthetic capping oligonucleotide 90 having an attached functional group 92. The capping oligonucleotide 90 is attached to the dsDNA 86 through ligation, thus creating a ligation site 100.

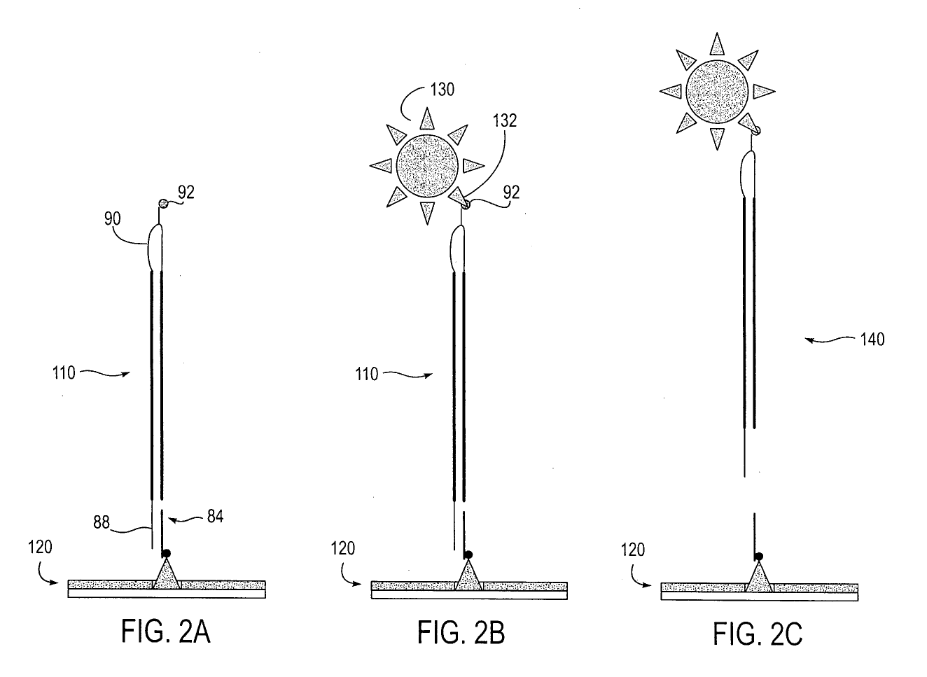

Referring now to FIG. 2, a method for providing a microsphere having a single target polymeric molecule attached is illustrated. In FIG. 2A, a modified dsDNA molecule 110 (such as that shown in FIG. 1B) having a synthetic capping oligonucleotide 90 containing an attached functional group 92, is attached to a modified substrate 120 through the hybridization of a surface-linked oligo-dT 84 to the dsDNA-linked oligo-dA 88. In FIG. 2B, a binding partner-coated microsphere 130 is attached to the modified dsDNA 110 through a linkage between the binding partner 132 and a functional group 92. Such an attachment can be created, for example, by contacting a binding partner-coated microsphere 130 in a fluid flow with the immobilized binding functional group-labeled dsDNA 110. Unattached microspheres can be washed from the substrate surface. Suitable surface-functionalized microsphere beads of varying sizes are commercially available (Bangs Laboratories, Inc., Fishers, Ind.). The microsphere beads typically have a diameter between about 0.1 microns (μm) and about 20 μm, preferably between about 0.5 μm and about 10 μm, and most preferably between about 1 μm and about 5 μm. The microsphere beads typically comprise materials such as polystyrene, glass, polysaccharides such as agarose, and latexes such as styrene butadiene.

FIG. 2B shows the release of a microsphere bead containing a single attached dsDNA molecule 140 from the substrate 120 surface through the melting of the oligo-dT oligo-dA hybridization attachment. Such a release can be accomplished, for example, by increasing the temperature or changing the pH as described further herein.

Video microscopy experiments have confirmed the immobilization of a single polymeric molecule to the substrate. For example, such video microscopy experiments have shown an agent, such as a microsphere bead, exhibiting Brownian motion within a confined location, thereby indicating the presence of a single polymeric molecule attached to the substrate. By detaching the polymeric molecule at the end attached to the substrate surface, a single molecule can be isolated by moving or transporting the agent, as previously described. Video microscopy experiments have further demonstrated the controlled release of the polymeric molecules from the substrate surface. Conventional microscopic techniques such as dark field microscopy, bright field microscopy, differential interference contrast microscopy, and fluorescent microscopy methods can also be used to demonstrate polymeric molecule immobilization and the controlled release of the polymeric molecules from the substrate surface.

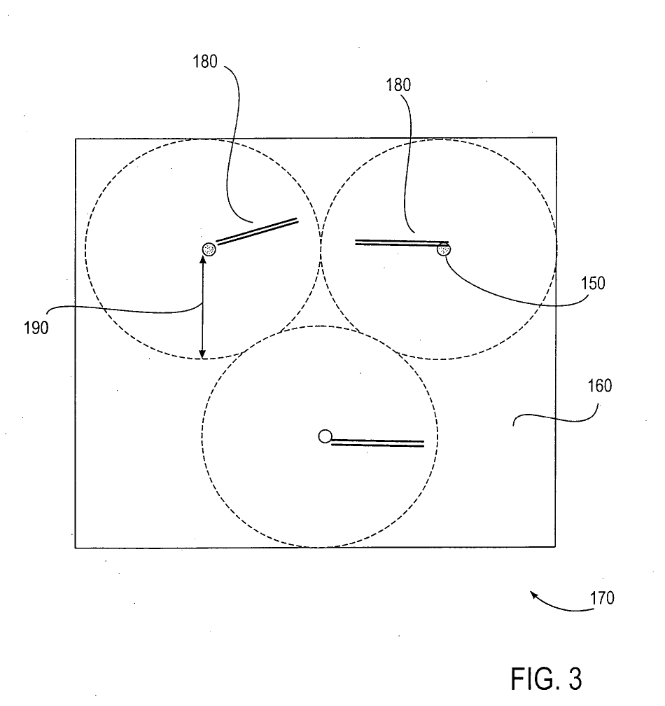

Embodiments of the invention provide substrate binding positions separated by at least two times the length of the target DNA molecule. A separation distance of at least two times the length of the target polymer molecule can prevent a microbead that is attached to an immobilized target polymer on a substrate surface from attaching to more than one immobilized target polymer. Substrate binding positions separated by at least about two times the length of the polymeric molecules can be provided by treating a substrate with a mixture comprising a binding agent and a blocking agent, the mixture having a ratio of binding agent to blocking agent of about one to about ten. The mixture ratio can also be about one binding agent to about 100 blocking agents, and about one binding agent to about 1000 blocking agents. Alternatively, the ratio can also be about one binding agent to about 10,000 blocking agents, one binding agent to about 100,000 blocking agents, one binding agent to about 1,000,000 blocking agents, and about one binding agent to about 10,000,000 blocking agents. Other suitable ratios can be determined by one having ordinary skill in the art. For example, referring to FIG. 3, a ratio of avidin 150 to BSA 160 coating a substrate 170 surface is calculated based on the length of a DNA target oligonucleotide 180. Specifically, a 5,000 nm length of DNA (assuming that 10,000 base pairs are 3,400 nm in length) 180, needs a 78,500 nm² area to be blocked out by BSA in order to provide binding positions for the target DNA molecules that are separated on average by two times the length of the target DNA 180. Thus the ratio of avidin 150 to BSA 160 in this example should be about 1 to 3,935,000 (or about 1 avidin 150 to about 4,000,000 BSA 160) (both avidin and BSA are about 65 kDa). In fact, since the effective binding sites of avidin will probably be fewer than 1 per bound avidin molecule, the avidin to BSA ration can be higher. Substrate binding positions separated by at least about two times the length of the polymeric molecules can be provided by treating the activated substrate first with a solution containing the binding agent and subsequently with a solution comprising the blocking agent. Binding positions separated by at least two times the length of the polymeric molecules are measured by the final effect achieved by the described surface treatment procedures, which are governed but not measured by the molecular ratios provided herein.

In one representative embodiment of the invention, the oligonucleotide label is biotin and the binding site attached to the substrate is avidin, and the oligonucleotide is immobilized at the precursor binding site position via the biotin moiety to form binding positions. In another representative embodiment of the invention, the oligonucleotide label is an antigen and the receptor attached to the substrate is an antibody for the antigen, and the oligonucleotide is immobilized at the precursor binding site position via the antigen to form binding positions. For example, the antigen can be digoxigenin and the antibody can be anti-digoxigenin antibody; the antigen can be fluorescein and the antibody can be anti-fluorescein antibody; and, the antigen can be cholesterol and the antibody can be anti-cholesterol antibody.

In additional embodiments of the invention, microsphere beads having a single target polynucleotide molecule attached are provided. In these embodiments, target polynucleotide molecules are attached to a microsphere bead. In general, the attachment of a target nucleic acid to a microsphere can be accomplished, for example, via binding-partner-functional group interactions. The microsphere bead is immobilized on a substrate surface through the hybridization of a single target polynucleotide molecule to a complementary polynucleotide that is attached to the surface, and single-stranded target polynucleotide molecules are digested. Microsphere beads having a single target polynucleotide molecule attached can then be released from the substrate surface. Such release can be controlled as disclosed herein.

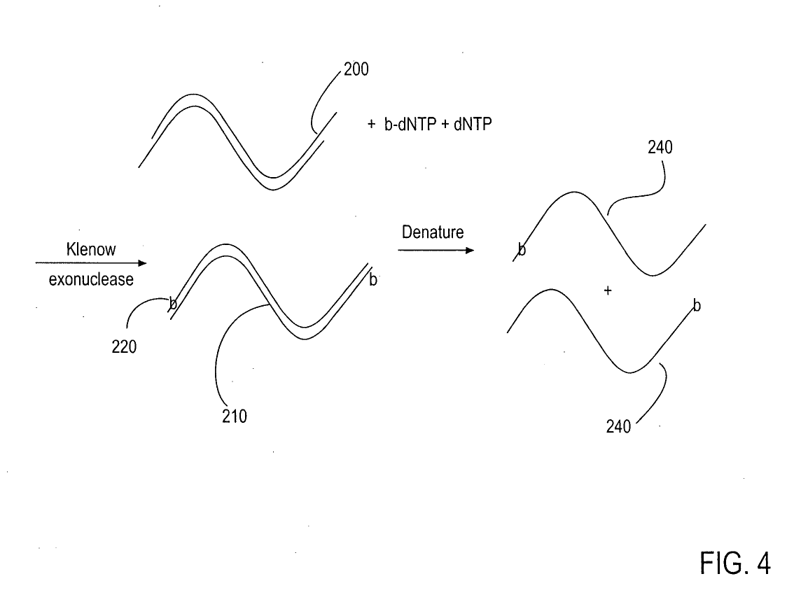

Referring to FIG. 4, nucleic acid molecules are modified on one end, for example, on or near either the 5′ ends or the 3′ ends, but not both the 5′ ends and the 3′ ends. In an exemplary embodiment, a double stranded DNA molecule (dsDNA) is ligated, for example, with a biotin-labeled double stranded linker, known in the art and commercially available, and subsequently denatured to provide two single stranded, end-labeled DNA molecules that are labeled on the 5′ or 3′ ends, but not both. Biotin-labeled dsDNA (labeled at either the 5′ ends or the 3′ ends, but not both the 5′ ends and the 3′ ends) can also be generated prior to ligation to include a biotin label on only one strand of the DNA, using for example, fill-in of overhanging ends in those instances where a single restriction enzyme has been used to digest the nucleic acid (thereby producing identical overhanging ends at the end terminus of the nucleic acid). In the example in FIG. 4, the Klenow fragment of DNA polymerase I is used to fill in the recessed 3′ ends of dsDNA fragments 200 with nucleotides from the reaction solution. Nucleotides in solution consist of unlabeled and biotin-labeled nucleotides (b-dNTP represents a biotin labeled nucleotide). The result is a double-stranded DNA having a biotin molecule 220 on the 3′ ends of the individual strands 210. Subsequent denaturation yields 3′-end-functional single-stranded DNA 240. Additionally, biotin-labeled dsDNA can be produced by ligating ‘linker’ DNA molecules, using a polymerase and biotin-labeled nucleotides, and carrying out fill-in reactions as described above. Similar procedures can be used for other functional groups. Functionalized nucleotides are commercially available, for example, from Molecular Probes (Eugene, Oreg.).

To create different modified ends of the DNA (partial single-stranded DNA termini), two different linkers with the same ligation sites can be used to ligate to the 5′ and 3′ ends generated by the same restriction enzyme. If different 5′ and 3′ ends are desired, two different restriction enzymes are used, and the DNA fragments are isolated based on size prediction, according to known information. In an alternative embodiment, the obtained single-stranded DNAs are labeled at both the 3′ (by polymerization or terminal transfer) and the 5′ ends (by ligation). The 3′ and 5′ ends can be labeled differently, for example one end with digoxigenin and the other end with biotin.

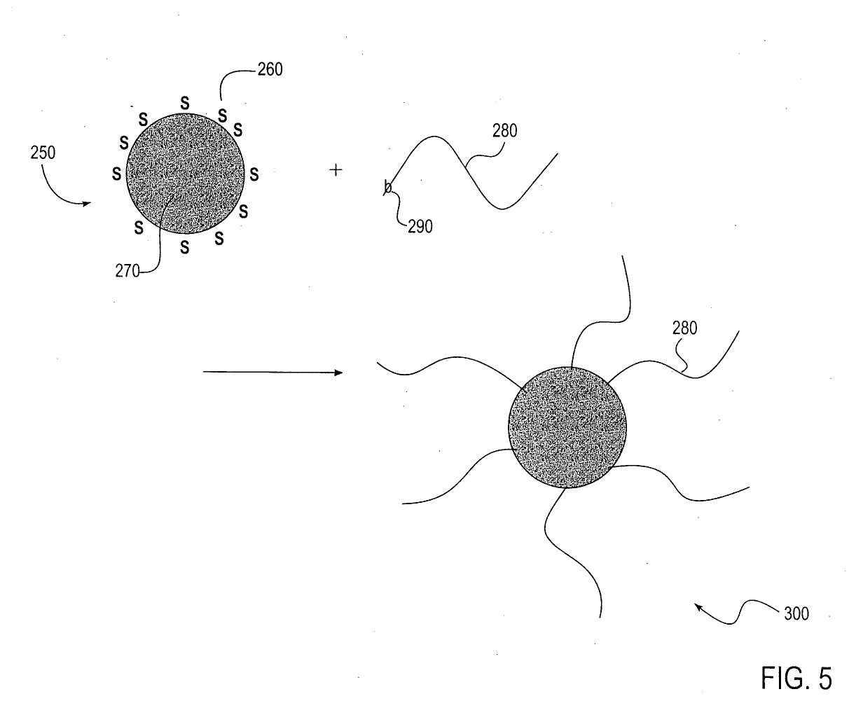

The single stranded, end-functionalized target DNA molecules are mixed with microsphere beads, under conditions that permit the formation of microsphere bead-polymeric molecule complexes. The DNA molecules may be provided in excess, such that there is more than one DNA molecule per bead. In an exemplary embodiment, the DNA molecules are functionalized and the microsphere beads have a coating comprising a binding partner having binding affinity for the functional group of the DNA molecule. For example, the DNA molecules can be functionalized with biotin and the binding partner can be microsphere beads coated with streptavidin (or alternatively, with avidin). Referring to FIG. 5, a streptavidin-coated microsphere 250 (comprised of streptavidin 260 and microsphere 270) is contacted with a solution containing a single-stranded DNA 280 having an attached biotin 290. The resulting microsphere 300 is now coated with several target DNA molecules 280. In another representative embodiment of the invention, the DNA is functionalized with an antigen and the binding partner attached to the microsphere bead is an antibody for the antigen. For example, the antigen is digoxigenin and the antibody is anti-digoxigenin antibody; the antigen is fluorescein and the antibody is anti-fluorescein antibody; and, the antigen can be cholesterol and the antibody can be anti-cholesterol antibody.

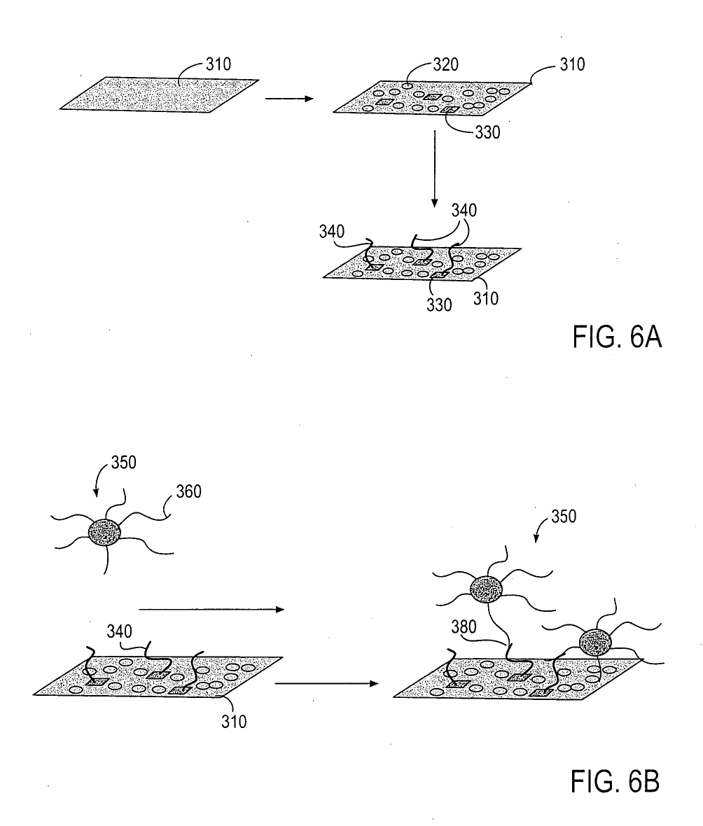

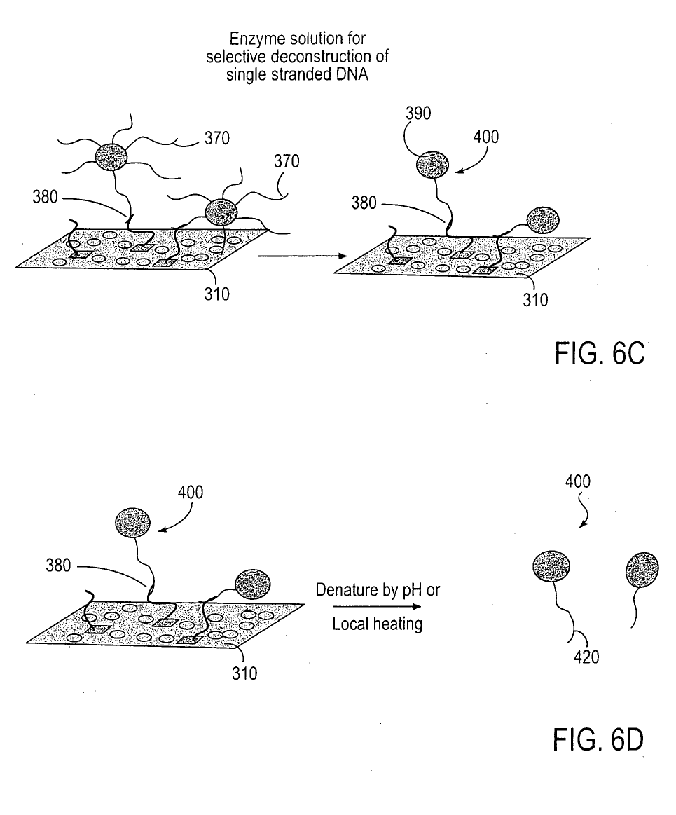

Referring now to FIGS. 6A-D, an embodiment of the invention that can be used to create microbeads having a single attached polymeric molecule is illustrated. FIG. 6A illustrates the attachment of a functionalized complementary oligonucleotide 340 to the surface of a substrate 310. In this example, the density of complementary oligonucleotides bound to the surface is controlled by coating the substrate 310 surface with both bovine serum albumen (BSA) 320 and avidin 330. A biotin-functionalized complementary oligonucleotide 340 is then attached to the substrate 310 through a biotin-avidin bond. Providing a substrate having binding positions (as described herein) separated by at least the length of the target DNA molecule prevents more than one strand of target DNA attached to the microbead from hybridizing to the complementary strands on the surface of the substrate. FIG. 6B illustrates the attachment of a microbead-target DNA complex 350 to the surface of a substrate. The microbead-DNA complex 350 is attached to the substrate 310 through the hybridization of a DNA strand 360 attached to the bead and a complementary oligonucleotide 340 attached to the surface. When the target single stranded DNA is labeled at the 5′ end, the immobilized oligomer should be complementary to the 3′ end of the target, and when the target single-stranded DNA is labeled at the 3′ end, the immobilized oligomer should be complementary to the 5′ end of the target. FIG. 6C illustrates the selective deconstruction of single-stranded DNA (ssDNA) 370 attached to a microbead 390 immobilized on the surface of the substrate 310. The ssDNA 370 is digested and the dsDNA 380 that attaches the microbead to the substrate through a DNA hybridization linkage remains intact. Selective deconstruction is accomplished, for example, by contacting an exonuclease-containing enzyme solution the substrate surface having the attached microbead-DNA complexes. The exonuclease can be 5′ specific if the 3′ end is labeled (and vice versa). Examples of suitable exonucleases, include, but are not limited to exonuclease 1, lambda exonuclease, or a DNA polymerase with exonuclease activity, such as T4 DNA polymerase or T7 DNA polymerase. Exonuclease 1 digests single stranded DNA from the 3′ to 5′ end; lambda exonuclease digests double stranded DNA from the 5′ to 3′ end; and T4 DNA polymerase (exonuclease) and T7 DNA polymerase (exonuclease) digest single and double stranded DNA from the 3′ to 5′ end. The result is a microbead 400 immobilized on a substrate 310 that has a single target DNA attached. FIG. 6D illustrates the release of microbeads 400 having only one ssDNA attached 420, from a substrate 310. The release is accomplished through the denaturation of the dsDNA linkage 410 through, for example, a change in pH or localized heating. Controlled molecule release of the target polymeric molecules can be achieved as described herein. According to an additional embodiment, the isolation of a microsphere bead having a single attached target oligomer can be performed in a microfluidic device. In this device, the substrate surface is part of a microfluidic channel.

In the methods according to one embodiment of the invention, microsphere beads have a coating comprising a binding partner having binding affinity for a functional group of the polymeric molecule. The functional group can be biotin and the binding partner can be either avidin or streptavidin. Alternatively, the functional group can be an antigen and the binding partner can be an antibody for the antigen, as previously described herein. For example, the functional group can be digoxigenin and the binding partner can be anti-digoxigenin antibody; the functional group can be fluorescein and the binding partner can be anti-fluorescein antibody; and, the functional group can be cholesterol and the binding partner can be anti-cholesterol antibody. Further examples of binding partners include protein-aptamer, lectin-sugar (and lectin-carbohydrate), and enzyme-inhibitor, cofactor, or substrate interactions.

In general, releasing a polymeric molecule immobilized to a surface through nucleic acid hybridization interactions can be effected by heating, adding a pH adjusting compound to the system, changing the salt concentration of the system or otherwise disrupting the hydrogen bonds formed between base pairs, for example by adding a disrupting agent such as guanidine salts, urea, dimethyl sulfoxide (DMSO), and/or formamide, which is capable of disrupting hydrogen bonds formed between base pairs. The heating temperature, pH change, salt concentration, and disrupting agent concentration will vary depending on the melting temperature of the hybridized first nucleotide/second nucleotide complex, but can be determined by well-known methods. Additionally, a restriction enzyme can be used if a portion of the polymeric molecule which is hybridized to the binding site oligomer on the substrate includes a restriction enzyme site.

The methods can further include transporting the at least one agent-polymeric molecule complex. According to one aspect, for example, an agent-polymeric molecule complex can be transported to a desired location using optical tweezers. According to another aspect, an agent-polymeric molecule complex can be transported by the flow of the fluid through the device.

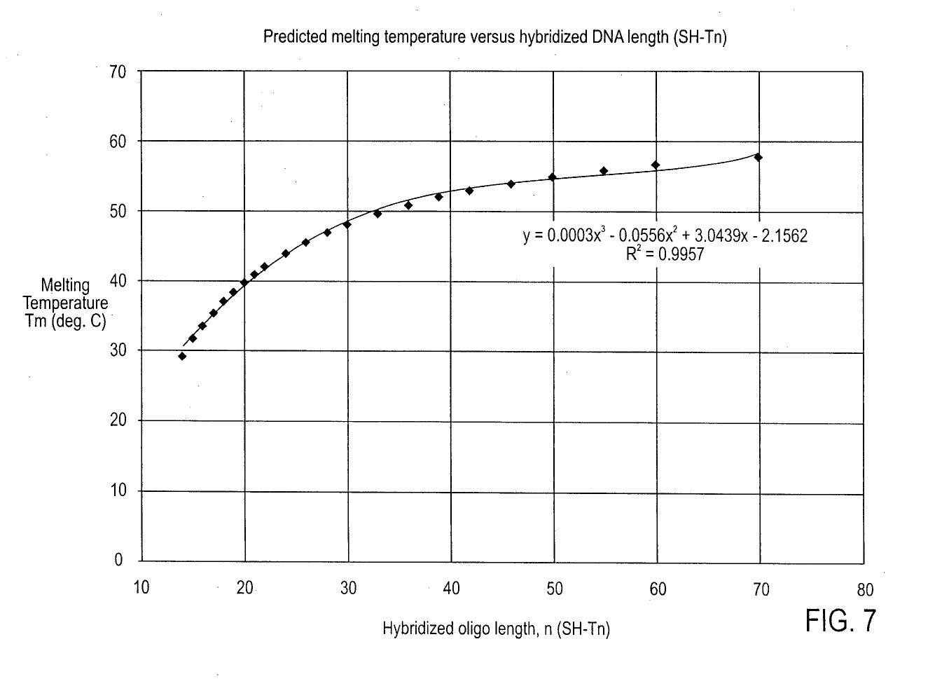

Referring now to FIG. 7, a graph is provided demonstrating the predicted calculated dependence of the melting temperature of double-stranded DNA on the length of the hybridized stand for an oligo-dA -dT hybridization. As can be seen, melting temperature increases with increasing length. Thus, in one embodiment of the invention, nucleotides containing an oligo-dA tail are controllably released from a surface containing varied hybridization lengths of oligo-dT at binding positions on the surface though heating the surface to which the polymers are attached in a controlled manner. For example, a surface having oligo-dT's providing hybridization lengths of about 15 to about 35 nucleotides is heated from about 35° C. to about 55° C. to controllably release the bound polymeric molecules. In general, the actual melting temperature of single molecule release correlates well with the expected theoretical melting temperature of the hybridized base pairs formed between the first oligonucleotide and the second oligonucleotide. Additionally, other complementary oligo sequences besides oligo-dA and oligo-dT can be used to provide hybridization lengths for attachment of polymeric molecules to a surface. Melting temperature can be determined either through well-known empirical methods, calculation, or a combination of both.

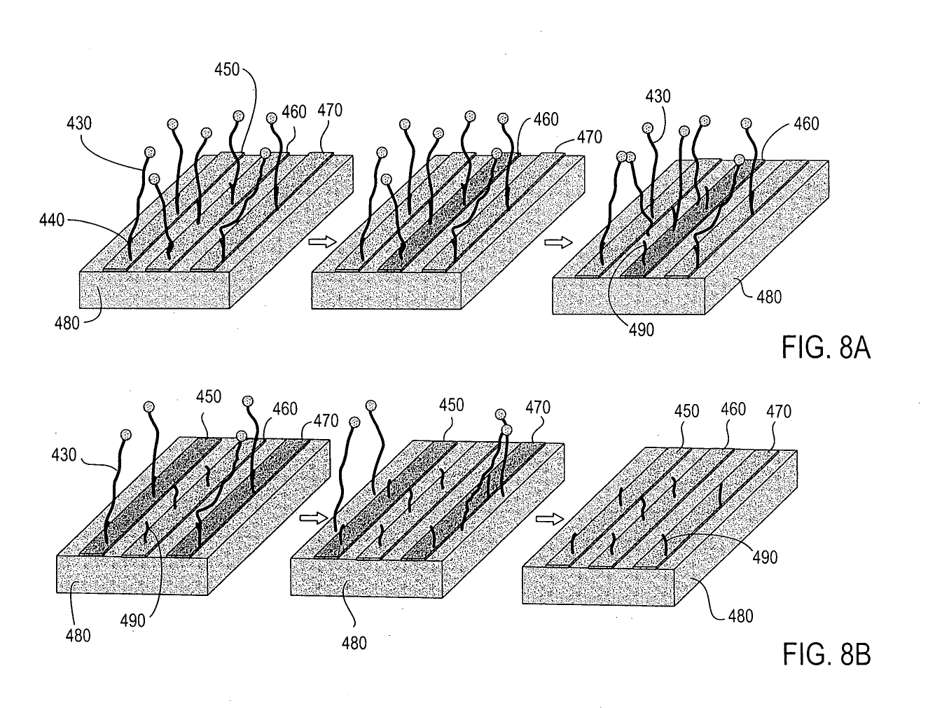

Referring now to FIG. 8, an illustration is provided of a method for controllably releasing polymeric molecules attached to a substrate. In this embodiment, the polymeric molecules 430 are hybridized to binding positions 440 above resistive heater strips 450, 460, and 470 on a section of a substrate 480. In FIG. 8A, the center resistive heater 460 is warmed releasing the polymeric molecules 430 closest to the center heater strip 460 through melting of the hybridized DNA 440. Left behind is the complementary DNA strand 490 that forms the hybridization binding site for the target DNA 430. In FIG. 8B, current is now passed though the outer electrodes 450 and 470 and the polymeric molecules hybridized above the outer electrodes 450 and 470 are released from the substrate 480 through melting of the hybridization bond 440 attaching them to the surface. Additionally, this method can be used with varying densities of target polymeric molecules attached to the surface and varying hybridization lengths of DNA as modes of attachment to achieve controllable release of polymeric molecules from the surface of the substrate. For example, the density of polymeric molecules is controlled as described herein so that several polymeric molecules are bound above a single resistive heater strip. By also varying the hybridization length attaching the polymeric molecules above the heater strip, and raising the temperature between, for example 30 and 55° C., polymeric molecules are released at different times as the temperature increases. Additionally, a resistive passivation layer may be placed between the heating elements and the fluid solution. Suitable passivation layers include, for example, silicon dioxide, silicon oxynitride, and silicon nitride layers deposited, for example, by plasma enhanced chemical vapor deposition (PECVD). Further, an additional layer containing gold, for example, may be deposited on the passivation layer to provide a substrate for biomolecule attachment. A biomolecule binding substrate layer may be patterned to allow for individually addressable substrate binding areas. For example, lines of heating elements aligned in one direction and lines of biomolecule binding substrate aligned in the perpendicular direction provide addressable release areas where the heating elements and the biomolecule binding substrate intersect.

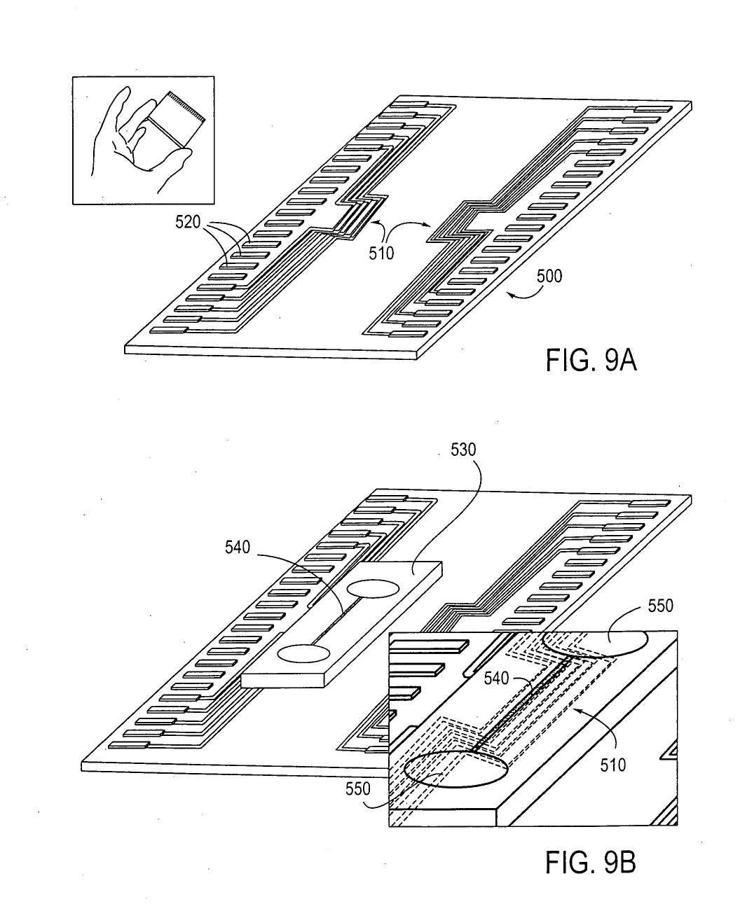

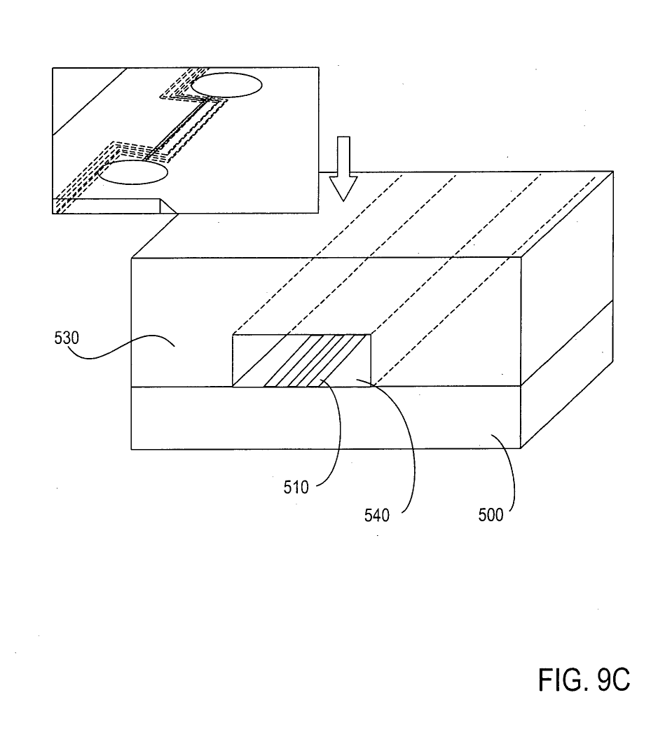

Referring now to FIGS. 9A-C, an example device that allows attached polymeric molecules to be controllably released is pictured. In this FIG. 9A, resistive heaters 510 have been placed on a glass substrate 500 in contact with gold contacts 520 that allow current to be delivered to the resistive heaters 510. In FIG. 9B a microchannel 540 formed in a PDMS (polydimethylsiloxane) block 530 having two reservoirs 550 has been placed above the resistive heating elements 510. Fluid solutions are flowed through the microchannel 530 to remove the polymeric molecules as they are released. FIG. 9C shows a further expanded view of the resistive heating elements 510 within the microchannel 540 formed in the PDMS block 530. Different patterns of heating elements and microchannels are possible and such a device may comprise an element in a larger microfluidic system.

Additionally, selective release of polymeric molecules attached to a substrate surface can be accomplished with microchannels that cross the substrate perpendicularly to the direction fluid flow across the substrate created by a second set of microchannel(s) that take released molecules to a collection device, for example. In this case, the flow of a solution that causes the attached polymeric molecules to release from the substrate, such as, for example, a solution that raises or lowers the pH, is at a higher temperature than the substrate, or varies the salt concentration of the solution above the substrate, can be selectively flowed through perpendicular microchannel(s) in contact with the substrate surface. Polymeric molecules are then selectively released from the substrate at the locations in which the fluid flow from the perpendicular channels contacts the substrate surface.

As used herein, the term hybridization or hybridize, refers to hybridization under moderately stringent or highly stringent conditions such that a nucleotide sequence preferentially associates with a selected nucleotide sequence over unrelated nucleotide sequences to a large enough extent to be useful in identifying the selected nucleotide sequence. It will be recognized that some amount of non-specific hybridization is possible, but is acceptable provided that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, for example, high stringency conditions, or each of the conditions can be used, for example, for 10-15 minutes each, in the order listed above. However, as mentioned above, optimal conditions will vary, depending on the particular hybridization reaction involved, and can be determined empirically.]

To minimize non-specific binding of the beads to the substrate, a solution containing a receptor, such as avidin, can be mixed in a 0.5 weight percent (wt. %) BSA solution. The device should subsequently be washed with a 0.5 wt. % BSA solution. The concentration range for the receptor containing solution, for example, a solution containing avidin, should be between about 0.01 nanomolar (nM) and about 10 nM. Approximately one to two milliliters (ml) of a receptor containing solution should be sufficient to provide precursor binding sites in a microfluidic device according to one embodiment of the invention (for example, a microfluidic device having a width of about 50 microns, and a length of about five centimeters).

In various embodiments of the invention, the arrays and substrates may be incorporated into a larger apparatus and/or system. In certain embodiments, the substrate may be incorporated into a micro-electro-mechanical system (MEMS). MEMS are integrated systems comprising mechanical elements, sensors, actuators, and electronics. All of those components may be manufactured by known microfabrication techniques on a common chip, comprising a silicon-based or equivalent substrate (See for example, Voldman et al., Ann. Rev. Biomed. Eng., 1:401-425, (1999).) The sensor components of MEMS may be used to measure mechanical, thermal, biological, chemical, optical and/or magnetic phenomena. The electronics may process the information from the sensors and control actuator components such as pumps, valves, heaters, coolers, and filters, thereby controlling the function of the MEMS.

The electronic components of MEMS may be fabricated using integrated circuit (IC) processes (for example, CMOS, Bipolar, or BICMOS processes). The components may be patterned using photolithographic and etching methods known for computer chip manufacture. The micromechanical components may be fabricated using compatible micromachining processes that selectively etch away parts of the silicon wafer or add new structural layers to form the mechanical and/or electromechanical components.

Basic techniques in MEMS manufacture include depositing thin films of material on a substrate, applying a patterned mask on top of the films by photolithographic imaging or other known lithographic methods, and selectively etching the films. A thin film may have a thickness in the range of a few nanometers to 100 micrometers. Deposition techniques of use may include chemical procedures such as chemical vapor deposition (CVD), electrodeposition, epitaxy and thermal oxidation and physical procedures like physical vapor deposition (PVD) and casting. Methods for manufacture of nanoelectromechanical systems may be used for certain embodiments of the invention. (See for example, Craighead, Science, 290: 1532-36, (2000).)

In some embodiments of the invention, substrates may be connected to various fluid filled compartments, such as microfluidic channels, nanochannels and/or microchannels. These and other components of the apparatus may be formed as a single unit, for example in the form of a chip, as known in semiconductor chips and/or microcapillary or microfluidic chips. Alternatively, the uniform substrates may be removed from a silicon wafer and attached to other components of an apparatus. Any materials known for use in such chips may be used in the disclosed apparatus, including silicon, silicon dioxide, silicon nitride, polydimethyl siloxane (PDMS), polymethylmethacrylate (PMMA), plastic, glass, quartz, and those having a gold surface layer, and the like.

Techniques for batch fabrication of chips are well known in the fields of computer chip manufacture and/or microcapillary chip manufacture. Such chips may be manufactured by any method known in the art, such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding with a flowable, optically clear material such as plastic or glass; photolithography and dry etching of silicon dioxide; electron beam lithography using polymethylmethacrylate resist to pattern an aluminum mask on a silicon dioxide substrate, followed by reactive ion etching. Methods for manufacture of nanoelectromechanical systems may be used for certain embodiments of the invention. (See for example, Craighead, Science, 290:1532-36, (2000).) Various forms of microfabricated chips are commercially available from, for example, Caliper Technologies Inc. (Mountain View, Calif.) and ACLARA BioSciences Inc. (Mountain View, Calif.).

According to an additional embodiment of the invention, a microfluidic device includes a micromold comprising a chemically inert material and having a top surface, a bottom surface, a sample inlet, a sample outlet, and a microchannel pathway defined between the sample inlet and the sample outlet, a substrate adhered to the bottom surface, the substrate having binding positions for immobilizing polymeric molecules, said binding positions separated by at least about two times the length of the polymeric molecules, and a heating element adapted to heat the substrate.

The micromold can comprise a silicone material. Typically, the microchannel has a width between about 10 microns and about 200 microns, and a length between about 0.25 centimeters and about five centimeters.

The binding positions can comprise a polymeric molecule. The substrate binding positions can be provided as described above. The polymeric molecule can comprise a thiol-modified oligonucleotide or a labeled oligonucleotide. The heating element can comprise a thin-film resistive heater. In one embodiment of the invention, the heating element is the substrate.

The microfluidic device can also further include a passivation layer between the substrate and the heating element. When the microfluidic device includes a passivation layer, a first pattern formed by the resistive heater can be different from a second pattern formed by the substrate. When the first pattern formed by the resistive heater differs from the second pattern formed by the substrate, the microfluidic device provides individually addressable binding positions, thereby facilitating the controllable release of an individual polymeric molecule adhered to the individually addressable binding position of the substrate. According to one aspect, the first pattern and the second pattern intersect at discrete locations to provide such individually addressable binding positions. According to a preferred embodiment of this aspect, the first pattern and the second pattern intersect at an approximately 90° angle.

According to another embodiment of the invention, a microfluidic device includes a micromold comprising a chemically inert material and having a sample well, a first end, a second end, and a microchannel pathway defined between the first end and the second end, and a first electrode disposed proximate to the first end and a second electrode disposed proximate to the second end. The inner surface of the microfluidic device can be modified such that it is neutral, negative or positively charged.

The microfluidic device can further include a collection chamber having a third end and a fourth, collection end, the collection chamber being substantially transverse to the microchannel. A third electrode can be disposed proximate to the collection chamber third end and a fourth electrode can be disposed proximate to the collection chamber fourth, collection end. The microfluidic device can further include a switching circuit between the first electrode and the second electrode. Further, the microfluidic device can further include a power supply operatively connected to the switching circuit.

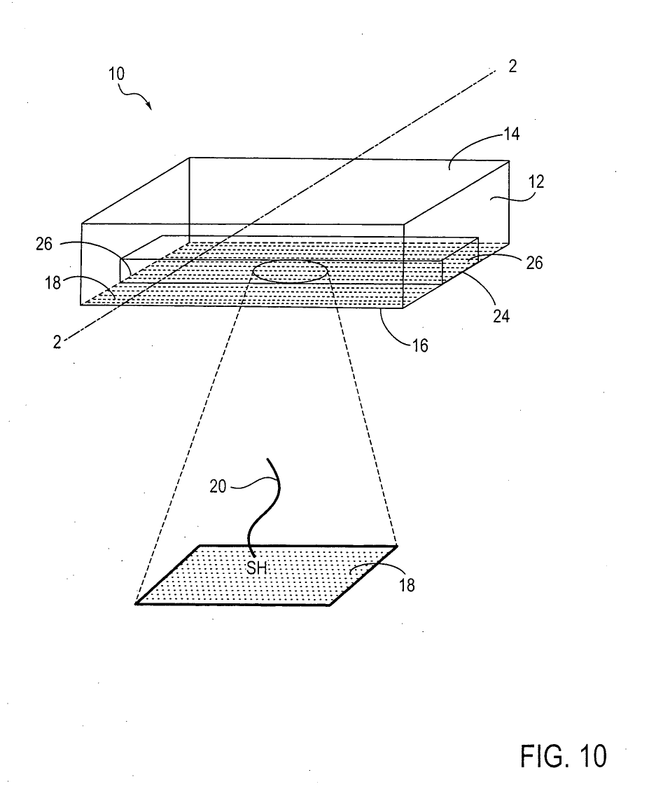

A microfluidic device in accordance with one embodiment of the invention is illustrated in FIG. 10. In FIG. 10 microfluidic device 10 includes micromold 12 and has a top surface 14 and a bottom surface 16. The microfluidic device also includes a substrate 18. Substrate 18 provides binding positions 20 (exemplified in the expanded, projected view as a thiol-modified oligomer immobilized to the substrate). Microfluidic device 10 further includes a sample inlet 22, a sample outlet 24, and a microchannel pathway 26 defined between the sample inlet and the sample outlet.

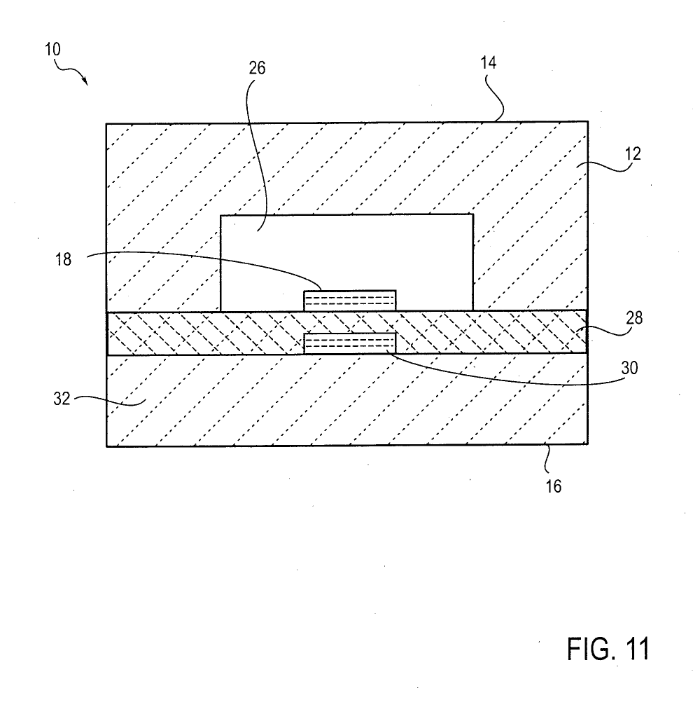

FIG. 11 shows a cross-sectional view of the microfluidic device 10 depicted in FIG. 10 along line 2-2. Microfluidic device 10 can include passivation layer 28. Further, microfluidic device 10 can include heating element 30 and supporting surface 32. Heating element 30 is depicted as a thin-film resistive heater. An electrical contact (not shown) is included to pass current through the heating element 30. A thermocouple (not shown) can be operatively connected to the heating element to measure the temperature at which a single molecule is released (heating temperature). A processing unit (not shown) can be used to program and control the heating temperatures. Additionally, other heating elements 30 can be used to release polymeric molecules immobilized to binding positions 20, including heating means such as a hot plate or a focused laser beam.

Passivation layer 28 serves to decouple substrate 18 from heating element 30. Passivation layer is often included to mitigate electrolysis problems that occur when substrate 18 is directly heated. Furthermore, controlled release of the immobilized polymeric molecules can be attained by decoupling substrate 18 and heating element 30, as is described in further detail below. Nonetheless, in one embodiment of the invention, the device 10 does not include a passivation layer 28, and the substrate 18 is also the heating element 30. Suitable passivation layers 28 include SU-8 photoresist, spin-on glass (SOG), plasma enhanced chemical vapor deposited (PECVD) silicone dioxide, and PECVD silicon oxynitride. Silicon oxide and silicon oxynitride layers are preferred and may be deposited by any conventional deposition technique, including chemical vapor deposition and thermal growth. The passivation layer 28 is typically at least about 1 micron thick. In an alternative embodiment, passivation layer 28 can be modified to provide binding sites for the substrate 18.

Techniques such as soft lithography and photolithography, which have been used in the semiconductor industry, can be used to fabricate micromold 12 of microfluidic device 10. For example, designs of micromold 12 were drawn to scale using CAD software. The designs were then printed onto transparencies using a high-resolution printer to form a transparency mask. “Photoresist on Silicon” masters for micromolding were prepared by standard photolithographic techniques using the transparency masks and a photoresist. These patterned masters were then silanized and used for micromolding with a silicone material such as poly(dimethyl siloxane) (PDMS). For example, PDMS precursor was poured onto the silanized master and then cured. The cured PDMS containing the channel structure was then bonded to the supporting surface 32 by applying pressure to enclose the channels. Typically, the microchannel pathways 26 were approximately 100 microns in width and between about two centimeters and about three centimeters in length.

The substrate 18 can also be prepared using standard lithographic techniques. For example, a photoresist can be deposited on substrate support surface 32 and exposed through a mask. The exposed photoresist can be developed. A suitable heating element 30 or substrate 18 material can be deposited by, for example, sputter deposition. In one embodiment, a thin layer of titanium or chromium having a thickness of about 80 Å is deposited, followed by subsequent deposition of a thin layer of gold having a thickness of about 240 Å. The photoresist is then lifted off of substrate support surface 32, thereby providing a substrate 18 and/or heating element 30 on the substrate support surface 32.

If a passivation layer 28 is to be incorporated into device 10, the initial structure formed on the substrate support surface 32 is a heating element 30, and a suitable passivation material can be deposited over the heating element 30 and over the substrate support surface 32 to form a passivation layer 28. Subsequently, a photoresist can be deposited on passivation layer

28 and exposed through a mask. The exposed photoresist can be developed. A suitable substrate 18 material can be deposited by, for example, sputter deposition. In one embodiment, a thin layer of titanium is deposited, followed by subsequent deposition of a thin layer of gold, as provided above. The photoresist is then lifted off of passivation layer 28, thereby providing a substrate 18 on the passivation layer 28. FIG. 18 shows a structure incorporating such a passivation layer 28. While FIG. 18 shows a structure wherein the deposition pattern of the heating element 30 is the same as the deposition pattern of the substrate 18, the pattern formed by the heating element 30 can be different from the pattern formed by the substrate 18, to provide an additional way of locally heating and releasing molecules immobilized to the substrate 18.

According to this aspect, a molecule immobilized to a binding position on the substrate can be individually addressed and controllably dispensed from the substrate surface by virtue of the different heating element and substrate patterns. For example, current applied to the heating element will only release those molecules immobilized at binding positions on the substrate that intersect with the heating element.

According to an additional embodiment of the invention, a method for isolating a single polymeric molecule includes introducing a mixture comprising microbead-polymeric molecule complexes having varying numbers of bound polymeric molecules into an applied electric field, and separating the microbead-polymeric molecule complexes having only one bound polymeric molecule from the mixture based on mobility. Separation of the mixture occurs because polymeric molecule (for example, nucleic acid) attachment to a microbead changes the charge of the formed microbead-polymeric molecule complex, therefore also its mobility in an applied electrical field.

The methods may further include determining the mobility of a microbead-polymeric molecule complex having only one bound polymeric molecule under the applied electric field. For example, the mobility of microbeads having no bound polymeric molecules can be easily measured. The ratio of polymeric molecules to carriers (microbeads) can be varied and the mobility distribution of the microbead-polymeric molecule complexes can be determined. Based on these data, the mobility of carrier with a single bound polymeric molecule can be predicted.

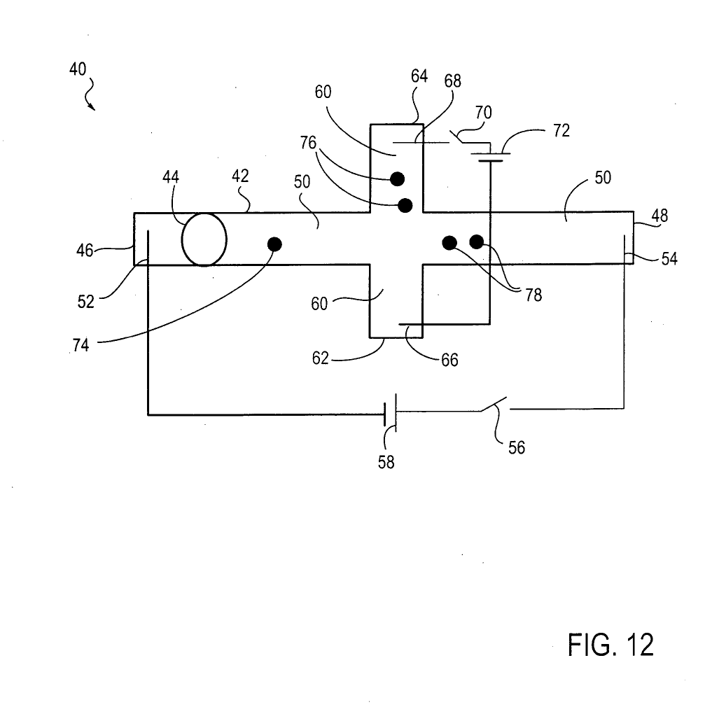

Referring now to FIG. 12, a microfluidic device in accordance with another embodiment of the invention is generally referred to by reference numeral 40. Microfluidic device 40 includes micromold 42. Micromold 42 includes a sample well 44, a first end 46, a second end 48, and a microchannel pathway 50 defined between the first end 46 and the second end 48. A first electrode 52 is disposed proximate to the first end 46 and a second electrode 54 is disposed proximate to the second end 48. A switching circuit 56 is located between the first electrode and the second electrode. Switching circuit 56 permits an applied field to be turned on and off. A power supply 58 is typically operatively connected to the switching circuit 56.

Microfluidic device 40 can include a collection chamber 60 having a third end 62 and a fourth, collection end 64. Typically, the collection chamber 60 is substantially transverse to the microchannel pathway 50. A third electrode 66 can be disposed proximate to the third end 62 and a fourth electrode 68 can be disposed proximate to the fourth, collection end 64. A switching circuit 70 is located between the third electrode 66 and the fourth electrode 68. Switching circuit 70 permits an additional field to be applied to the collection chamber, thereby facilitating separation of the desired polymeric-agent complexes. A power supply 72 is typically operatively connected to the switching circuit 70.

FIG. 12 further shows the application of a method in accordance with one embodiment of the invention. For example, FIG. 12 shows the separation of agent-polymeric molecule complexes having only one bound polymeric molecule from a mixture comprising agent-polymeric molecule complexes having varying numbers of bound polymeric molecules in an applied electric field. In FIG. 12, an agent having no bound polymeric molecules is depicted as reference number 74, two agent-polymeric molecule complexes having only one bound polymer are depicted as reference number 76, and two agent-polymeric molecule complexes having more than one bound polymer are depicted as reference number 78. An initial applied field between the first end 46 and the second end 48 results in an initial separation of the mixture. When the desired polymeric-agent complexes 76 (i.e., those agent-polymeric molecule complexes having only one bound polymeric molecule) have migrated to the collection chamber 60, switching circuit 56 can be turned off such that the field applied between the first end 46 and the second end 48 is no longer applied. Switching circuit 70 can then be turned on to promote movement of the desired polymeric-agent complexes 76 towards the collection chamber, collection end 64, to isolate the single polymeric molecule.

### EXAMPLES

### EXAMPLE 1

Target Polymeric Molecule Preparation

Modified λ-phage DNA (48.5 kbps) was used as the target DNA in this study. λ-phage DNA was modified through ligation using DNA oligomers such that one end of the DNA had a complementary sequence that hybridizes to a substrate binding site oligomer, and the other end had a biotin label for attachment to an agent (for example, a polystyrene (PS) bead). After ligation, modified λ-DNA molecules were separated from the short DNA oligomers by adding polyethylene glycol to cause the precipitation of the modified λ-DNA molecules. Precipitated target DNA was collected and dissolved in buffer and stored at 4° C. before use.

Specifically, for 200 microliters (μl) ligation reaction, 40 μl of stock solution of lamba-phage DNA (0.5 ng/μl), 10 μl of 10 μM LcosA30 (an exemplary oligomer to be ligated to the 5′ overhang of the lambda-DNA having the following sequence: 5′ pAGG TCG CCG CCC AAA AAA AAA AAA AAA AAA AAA AAA AAA AAA 3′), 10 μl of 10 μM Rcos (an exemplary oligomer to be ligated to the 3′ overhang of the lambda-DNA having the following sequence: 5′ pGGG CGG CGA CCT AAA TTT ATA TTT TTT T[B]T TTT TTT TAT AAA TTT 3′), and 20 μl of 10× ligase buffer were mixed together gently (after adjusting the volume to 200 μl with water) and heated to 65° C. for 10 min. 4 μl of T4 DNA ligase was added to the ligation reaction mixture after the mixture was cooled down to approximately room temperature (˜25° C.). The ligation reaction mixture was then stored at room temperature for about 9 to about 15 hours such that the ligation reaction could proceed to completion.

In order to separate the short oligomers from the modified lambda-DNA, precipitation using poly(ethylene glycol) (PEG) was performed. According to this procedure, equal volumes of solutions containing 20 wt. % PEG and 2M NaCl were added to the modified λ-DNA solution. The resulting solution was mixed gently until the modified λ-DNA precipitated from solution. The supernatant solution was then removed by centrifugation and discarded. The DNA pellets were resuspended in 1× TE buffer (10 mM Tris HCl, pH 7.8 and 1 mM EDTA) to form a DNA solution. The remaining PEG and NaCl in the DNA solution were removed after adding ethanol to provide a 70% ethanol solution (by volume), thereby precipitating the modified lambda-DNA again. Finally, the DNA pellets were resuspended in 1× TE buffer. Each of the resultant modified λ-DNA molecules is expected to have a single-stranded region at one end and a biotin label at the other end.

The following procedure was used to conduct static (no flow) experiments:

Substrate Modification

A 1 micromolar solution of thiol-modified DNA oligomer (Qiagen-Operon, Valencia, Calif.) was pipetted onto the surface of a gold thin film substrate and incubated at room temperature for 3-4 hrs. The surface was then washed with phosphate buffer saline (1×PBS) several times to remove unbound oligomers.

Immobilization of Target DNA

A 10 nanomolar solution of target DNA dissolved in 1×PBS was pipetted onto the substrate and incubated at room temperature for 1-2 hrs. After immobilization by hybridization, the substrate was then rinsed with 1×PBS more than three times to remove unhybridized DNA molecules.

Formation of Agent-Polymeric Molecule Complexes

After immobilization, a solution of streptavidin-coated polystyrene (PS) beads (1 μm diameter; 1:10 dilution of original solution in PBS obtained from Polysciences, Inc.) was incubated on the substrate for 1 hr to attach the beads to the biotinylated end of the target λ-DNA. The beads allowed the molecules to be visualized, and served as handles for optical manipulation after release of the polymeric molecule from the substrate.

For the dynamic (within microfluidic channels) experiments:

The reagents were pumped through the channels in the same order as in the static case, for 5 min by applying vacuum, followed by incubation within the microfluidic channels for time periods comparable to those used in the static experiments.

Single Molecule Isolation in Static Conditions

The density of single target DNA molecules hybridized using static conditions was 3-5 molecules per 100 μm×100 μm square area. The beads attached to target DNA exhibited Brownian motion but were restrained to within a radius of about two to three microns. DNA immobilization and bead attachment were further confirmed by using a standard upright optical microscope equipped with optical tweezers by trapping and pulling the beads attached to the target DNA molecules.

Single Molecule Isolation within Microfluidic Channels

A microfluidic device in accordance with one embodiment of the invention permitted easy identification of the DNA/bead complexes that were immobilized. The efficiency of hybridization within microfluidic flows was lower than in the static case as expected, facilitating single molecule isolation at multiple dispersed locations on the substrate. The number of single molecules isolated within a microfluidic channel of 100 μm width and 1 cm length was approximately 10-20 molecules.

Single Molecule Release within Microfluidic Channels by Electrical Heating

After visualization of single molecule immobilization, single molecule release was achieved by heating the chip. Heating was performed using a thin film resistive heater located underneath the chip and controlling the current passing through the resistive heater. When the local temperature at the binding position on the substrate exceeds the melting temperature of the hybridized DNA molecule, the hybridized DNA molecule denatures and is released from the substrate.

For the DNA sequences that were chosen, the theoretical release temperature was 48.9° C. The releases of various single DNA molecules isolated were observed at substrate temperatures ranging from 46° C. to 53° C. This range was observed for single molecules released from the same microfluidic channel, as well as from multiple channels.

### EXAMPLE 2

Substrate Modification

A glass surface is treated with alkaline solution (NaOH, 1N) to expose hydroxyl groups. The hydroxylated surface is subsequently treated with an aldehyde-containing silane reagent (10 millimolar in 95% ethanol) to provide an aldehyde-activated substrate. After washing with ethanol three times, and deionized water three times, the aldehyde-activated substrate is coated with a solution containing avidin and BSA (bovine serum albumin) in certain molar ratio: 1:10 or 1:1000, etc. The aldehydes react readily with primary amines on the proteins to form Schiff's base linkages between the aldehydes and the proteins, i.e., to covalently attach the proteins to the aldehyde-activated substrate surface.

A poly-(dT)30 oligonucleotide can be obtained commercially (Qiagen-Operon). The 5′ end is labeled with a biotin moiety. The oligonucleotide is allowed to bind to the coated glass surface, followed by washing/cleaning to remove free (non-attached) oligonucleotide molecules with 1×PBS.

Agent Preparation

Streptavidin coated micro-sphere (fluorescent) of 1 um can be purchased from a commercial source (Polysciences Inc.)

Target Molecule Preparation

A DNA sample is digested with two different restriction enzymes to create DNA fragments having two different ends (for example, 10 micrograms of yeast DNA is digested in 100 microliters of 1× restriction enzyme digestion buffer (New England Biolabs), containing 50 units of EcoR1 and 50 units of BamH1). About 10 nanograms of a 20 kbp DNA fragment are isolated from agarose gel by methods known by those of ordinary skill in the art. A hairpin-like oligonucleotide (cap-oligo) with a biotin moiety in the middle and a restriction enzyme site at its end is synthesized and ligated to the desired end (determined by the restriction enzyme). After ligation, the DNA has a closed end with a biotin and an open end.

50 microliters of an enzyme solution containing terminal transferase (20 units) and 10 micromolar dATP can be used to add a poly dA tail (20-50 nucleotides long) to the open end of the DNA. Other end modification methods can also be used, depending on the final application of the molecule.

Single DNA Molecule Isolation

The target DNA is added to the substrate, and the oligo-dA tail hybridizes to the poly-dT nucleotide attached to the substrate surface in a standard hybridization buffer (1×PBS) that maintains proper pH and salt concentrations. The substrate is cleaned with 1×PBS buffer to remove free target molecules. The immobilized target molecules agents are contacted with an agent, here streptavidin coated microspheres to localize the target DNA molecules. The immobilized microspheres can be located with a microscope. Immobilization occurs when streptavidin binds to the biotin moiety in the DNA.

The presence of single DNA attaching to a carrier can be confirmed by an optical trapping technique. For example, an optical tweezers can stretch a DNA molecule by moving the carrier in different directions. The maximum stretching range (about 15 um for lambda DNA) and force applied can be measured and used as indicators for the presence of single molecule because the presence of more than one molecules will result in smaller stretching range for a given force.

To isolate a particular DNA molecule, a laser beam can be applied to the position where there is an immobilized microsphere, the local heating effect generated by the laser can denature the poly-dA and poly-dT hybrid, and thus release the DNA molecule (which is still attached to the microsphere) from the substrate surface. The DNA molecule is isolated by transporting the microsphere to a desired location using optical tweezers.

### EXAMPLE 3

Target Molecule Preparation

The ends of target DNA molecules are labeled with an appropriate functional group (for example, by using enzymes such as Klenow fragment and biotin-labeled nucleotides, if using streptavidin-coated beads) such that the labeled DNA molecules can bind to the surface of the beads. Labeling typically is performed on a dsDNA molecule, and then the strands are separated. A linker can be ligated to the 5′ end of the top strand of the double-stranded DNA and a poly dA tail can be added to the 3′ end of the same strand. Single stranded molecules having both ends modified can be obtained after denaturing the DNA.

Alternatively, the single-stranded DNA could be labeled at the 3′ (by polymerization or terminal transferase reaction) end or the 5′ end (by ligation). The 3′ and the 5′ ends can be labeled differently, for example, one end with digoxiginin and the other with biotin.

Formation of Agent-Polymeric Molecule Complexes

The end-labeled single stranded DNA molecules are mixed with surface-functionalized beads (for example, microspheres coated with streptavidin) such that the DNA strands are in excess (i.e., there is more than one DNA molecule per bead). The end-labeled single-stranded DNA binds to the coated surface of the microspheres.

Substrate Modification

The surface of a substrate is functionalized with appropriate DNA oligonucleotides so that the attached oligomers can hybridize with at least a portion of the target, single-stranded DNA molecule of interest.

The density of the oligomers attached to the substrate surface is controlled by first contacting the substrate with different ratios of avidin and bovine serum albumin (BSA), for example, 1:1000. The biotin-labeled oligomers are subsequently attached to the avidin through a biotin-avidin linkage.

Immobilization of Agent-Polymeric Complex to Substrate

The oligomer modified surface is contacted with the DNA-bead complex solution (for example, by flowing the solution over the substrate). At least one of the DNA single-stranded molecules on the bead hybridizes with the oligomer attached to the substrate surface.

Removal of Polymeric Molecules on the Agent-Polymeric Molecules which are not Immobilized to the Surface

The substrate surface is contacted with an enzyme solution (for example, by flowing, etc.) to selectively deconstruct the single stranded DNA molecules on the surface of the bead while leaving the bead attached to the surface. In this case, an enzyme solution comprising exonuclease can be used. The enzyme solution should be 5′ or 3′ specific depending on the labeling of the target and the capture DNA (5′ specific if the single strand target DNA is labeled at the 3′ end and vice versa).

Single Molecule Release

The substrate surface is contacted with a solution (for example, by flowing, etc.) to denature the hybridization between the oligomer attached to the substrate surface and the single-stranded DNA molecule of interest, which is attached to the bead, thereby causing the beads (and the target molecule of interest) to be released. Local heating of the surface can be performed such that the temperature exceeds the melting temperature of the hybridization to release the beads.

### EXAMPLE 4

Target Molecule Preparation

For Labeled DNA:

A DNA sample is digested with two different restriction enzymes to create DNA fragments having two different ends. For example, 10 micrograms of yeast DNA is digested in 100 microliters of 1× restriction enzyme digestion buffer (New England Biolabs), containing 50 units of EcoR1 and 50 units of BamH1. About 10 nanograms of a 20 kbp DNA fragment are isolated from agarose gel by methods known in the art. A hairpin-like oligonucleotide (cap-oligo) with a biotin moiety in the middle and a restriction enzyme site at its end is synthesized. The cap oligo is ligated to the desired end of the target molecule (determined by the restriction enzyme, for example EcoR1). After ligation, the target DNA has a closed end with a biotin and an open end.

For Tailed DNA:

Terminal transferase can be used to add a poly dA tail (20-50 nucleotides long) to the ends of a DNA molecule. For example, 50 microliters of an enzyme solution containing 20 units of terminal transferase and 10 micromolar dATP can be used to add a poly dA tail between about 20 and about 50 nucleotides long.

Formation of Agent-Polymeric Molecule Complexes

For Labeled DNA:

Streptavidin coated microspheres (fluorescent) can be obtained from a commercial source (Polysciences Inc). About 1 microgram of biotin-labeled DNA molecules is mixed with the microspheres (carriers) in a 1 molecule to 1 agent ratio in a binding buffer (1×PBS plus 0.1% Tween-20). The unbound DNA molecules are removed using centrifugation at 14,000×g for 10 min. The pellet is resuspended in the binding buffer (1×PBS plus 0.1% Tween-20) and the washing procedure (resuspending the bead-DNA complexes in binding buffer and centrifugation) is repeated two more times. Finally, the agent-polymeric molecule complexes (here, bead-DNA complexes) are resuspended in 50 microliters of the same binding buffer.

For Tailed DNA:

Streptavidin coated microspheres (fluorescent) can be obtained from a commercial source (Polysciences Inc). Mix biotin-labeled oligomer dT (1 micromolar in 1×PBS, plus 0.1% Tween-20) with the micro-spheres (carriers), and remove unbound oligonucleotides by centrifugation and washing, as described above. The tailed target DNA molecules can hybridize to the oligomer dT (on the agents) in a 1 molecule to 1 agent ratio in 50 microliters of the same buffer. The unbound DNA molecules are removed using centrifugation, as described above.

Single DNA Molecule Isolation According to Carrier Mobility

The carrier-DNA complexes are introduced to the sample well of a microfluidic device in accordance with an embodiment of the invention. A voltage is applied to separate the carriers along the length of the microchannel pathway. Agents having a single bound polymeric molecule can be isolated based on a predicted mobility corresponding to single DNA molecule attachment by directing the carrier to a collection chamber/channel using an additional applied electrical field and/or fluidic pressure/vacuum.

1) A method for isolating a target nucleic acid molecule on a substrate comprising:
a) hybridizing a section of the target nucleic acid molecule to a second nucleic acid molecule attached to a substrate at a binding position on the substrate, wherein binding positions on the substrate are separated by at least two times the length of a target nucleic acid molecule;
b) removing any unhybridized target nucleic acid molecules;
c) contacting the substrate with a solution containing microsphere beads having a binding partner capable of attaching to a label on the target nucleic acid molecule under conditions that allow the microsphere beads to attach to the label of target nucleic acid hybridized to the substrate surface; and
d) removing any unattached microsphere beads.

2) The method of claim 1 wherein the nucleic acid is a deoxyribonucleic acid.

3) The method of claim 1 wherein the substrate is comprised of a material selected from the group consisting of gold, aluminum, silicon, glass, and polymers.

4) The method of claim 1 wherein the label on the target nucleic acid molecule is biotin and the binding partner is selected from the group consisting of avidin and streptavidin.

5) The method of claim 1 wherein the label is an antigen and the binding partner is an antibody for the antigen.

6) The method of claim 1 further including releasing the target molecule from the substrate surface.

7) The method of claim 6 wherein the releasing is accomplished by changing the pH of a solution in contact with the substrate, changing the salt concentration of a solution in contact with the substrate, heating the substrate, or contacting the target nucleic acid molecules with a solution containing a restriction enzyme.

8) The method of claim 1 further including controllably releasing the target nucleic acid molecules from the substrate surface, wherein the controllably releasing is accomplished by selectively heating a portion of the substrate containing bound target polymeric molecules.

9) A method for providing a microsphere bead having a single target nucleic acid molecule attached comprising:
a) contacting a labeled target nucleic acid with a microsphere bead having a binding partner capable of binding to the label of the target nucleic acid molecule under conditions that allow the label of the target nucleic acid molecule to attach to the binding partner of the microsphere bead;
b) hybridizing a section of the target nucleic acid molecule to a second nucleic acid molecule attached to a substrate at a binding position on the substrate, wherein the binding positions on the substrate are separated by at least two times the length of the target nucleic acid molecule;
c) removing any microsphere beads that are not attached to the substrate; and
d) digesting any unhybridized nucleic acid molecules attached to the microsphere bead.

10) The method of claim 9 wherein the substrate is comprised of a material selected from the group consisting of gold, aluminum, silicon, glass, and polymers.

11) The method of claim 9 wherein the label is biotin and the binding partner is selected from the group consisting of avidin and streptavidin.

12) The method of claim 9 wherein the label is an antigen and the binding partner is an antibody for the antigen.

13) The method of claim 9 further comprising releasing a microbead containing a single nucleic acid molecule from the substrate.

14) The method of claim 13 wherein the releasing is accomplished by changing the pH of a solution in contact with the substrate, changing the salt concentration of a solution in contact with the substrate, heating the substrate, or contacting the target nucleic acid molecules with a solution containing a restriction enzyme.

15) A method for isolating microsphere beads having a single attached polymeric molecule comprising:
a) introducing a mixture comprising microsphere bead-polymeric molecule complexes into an applied electric field, said mixture including microsphere bead-polymeric molecule complexes having varying numbers of polymeric molecules bound to the microsphere beads; and
b) separating the agent-polymeric molecule complexes having only one bound polymeric molecule from the mixture based on mobility to isolate a single polymeric molecule.

16) The method of claim 15 wherein the polymeric molecule is a nucleic acid.

17) The method of claim 15 wherein the polymeric molecule is a deoxyribonucleic acid.

18) A microfluidic device for manipulating target nucleic acid molecules comprising:
a) a chemically inert housing having a bottom surface, a fluid inlet and a fluid outlet;
b) the housing also having at least one microchannel pathway defined between the sample inlet and the sample outlet wherein at least a portion of the microchannel is formed in the bottom surface of the housing;
c) a substrate adhered to the bottom surface, the substrate having binding positions for immobilizing target nucleic acid molecules, the binding positions separated by at least about two times the length of a target polymeric molecule and wherein a target nucleic acid molecule is from about 500 to about 50,000 nucleotides in length; and
d) a heating element adapted to heat the substrate.

19) The microfluidic device of claim 18 wherein the housing comprises a silicone material.

20) The microfluidic device of claim 18 wherein the microchannel has a width between about 10 microns and about 200 microns.

21) The microfluidic device of claim 18 wherein the microchannel has a length between about 0.25 centimeters and about five centimeters.

22) The microfluidic device of claim 18 wherein the binding positions comprise a polymeric molecule.

23) The microfluidic device of claim 22 wherein the polymeric molecule comprises a thiol-modified oligonucleotide.

24) The microfluidic device of claim 18 wherein the heating element comprises a thin-film resistive heater.

25) The microfluidic device of claim 18 further comprising a passivation layer between the substrate and the heating element.

26) The microfluidic device of claim 25 wherein a first pattern formed by the resistive heater is different from a second pattern formed by the substrate.

27) The microfluidic device of claim 26 wherein the first pattern and the second pattern intersect at locations, thereby providing individually addressable binding positions.

28) A device for controllable release of target nucleic acid molecules comprising,
a) a substrate having a surface;
b) a patterned heating element disposed on the substrate surface;
c) a passivation layer disposed on the heating element; and
d) a patterned layer adapted to bind target nucleic acid molecules disposed on the passivation layer wherein the patterned layer adapted to bind target nucleic acid molecules contains target nucleic acid molecule attachment sites separated by at least two times the length of a target nucleic acid molecule and wherein a target nucleic acid molecule is from about 500 to about 50,000 nucleotides in length.

29) The device of claim 28 wherein the substrate is comprised of a material selected from the group consisting of glass, silicon, aluminum, or polymer.

30) The device of claim 28 wherein the patterned layer is comprised of glass, gold, polymer, or silicon.

31) The device of claim 28 wherein the patterned heating element is a thin-film resistive heater.

32) The device of claim 28 wherein a pattern formed by the patterned heating element is different from a pattern formed by the patterned layer adapted to bind target nucleic acid molecules.


SEQUENCE LISTING (2 sequences)

SEQ ID NO: 1 - DNA, 42 bp, Artificial Sequence
  Synthetic sequence useful in the invention.
aggtcgccgcccaaaaaaaaaaaaaaaaaaaaaaaaaaaaaa

SEQ ID NO: 2 - DNA, 45 bp, Artificial sequence
  Synthetic sequence useful in the invention.
gggcggcgacctaaatttatatttttttbttttttttataaattt